(19) United States
(12) Patent Application Publication
(10) Pub. No.: US 2018/0125400 A1
(43) Pub. Date: May 10, 2018

(54) GLUCOSE MEASURING DEVICE AND APPARATUS

(71) Applicant: APEX BIOTECHNOLOGY CORP., Hsinchu (TW)

(72) Inventors: Mon-Wen Yang, Hsinchu (TW); Ying-Che Huang, Hsinchu (TW)

(73) Assignee: APEX BIOTECHNOLOGY CORP., Hsinchu (TW)

(21) Appl. No.: 15/434,080

(22) Filed: Feb. 16, 2017

(30) Foreign Application Priority Data
Nov. 4, 2016 (TW) 105135834

(51) Int. Cl. A61B 5/145; A61B 5/1477; A61B 5/1491; A61B 5/1486

(52) U.S. Cl. CPC A61B 5/14532; A61B 5/14507; A61B 5/1486; A61B 5/1491; A61B 5/1477

(57) ABSTRACT

A glucose measuring device and apparatus are provided. The device includes a substrate, a cover plate, an electrode assembly, and a reactive unit. The substrate has a first and second surfaces opposite to each other, and a flow channel located at the first surface. The flow channel includes a sampling region having a sample inlet, a measuring region, and a concentrating region therebwtween. The cover plate having a gas outlet is disposed on the first surface and covers the flow channel. The electrode assembly includes a first, a second and a third electrode pairs. The first electrode pair is at a boundary between the sampling and the concentrating regions. The second electrode pair is at a boundary between the concentrating and the measuring regions. The third electrode pair is located in the measuring region. The reactive unit is disposed on the third electrode pair and in the flow channel.

### CROSS-REFERENCE TO RELATED APPLICATION

This application claims the priority benefit of Taiwan application no. 105135834, filed on Nov. 4, 2016. The entirety of the above-mentioned patent application is hereby incorporated by reference herein and made a part of this specification.

### BACKGROUND OF THE INVENTION

### Field of the Invention

The invention relates to a glucose measuring device and apparatus, and particularly relates to a noninvasive glucose measuring device and apparatus.

### Description of Related Art

Until now, there are a number of methods and devices for monitoring and measuring glucose in blood of humans or animals. However, these methods are usually invasive techniques. That is, they will cause trauma to humans or animals. Thus, they have a certain degree of risk, or easily cause humans or animals to feel uncomfortable in the process of using.

Recently, some noninvasive glucose measuring devices have been developed on the market, such as optical noninvasive glucose detecting devices, or tear glucose detecting devices. However, these noninvasive glucose detecting devices have problems with high cost and lack of accuracy.

Additionally, a number of academic studies have found that glucose content in blood has a correlation with glucose content in saliva. However, the glucose content in the saliva is only one percent to one-tenth of the glucose content in the blood. Thus, the glucose content in the saliva can not be measured accurately by current technology.

### SUMMARY OF THE INVENTION

The invention provides a glucose measuring device having better measurement accuracy of glucose concentration.

The invention provides a glucose measuring apparatus having the aforementioned glucose measuring device.

The invention provides a glucose measuring device including a substrate, a first cover plate, an electrode assembly, and a reactive unit. The substrate has a first surface and a second surface opposite to each other, and a flow channel located at the first surface. The flow channel includes a sampling region having a sample inlet, a concentrating region, and a measuring region. The concentrating region is located between the sampling region and the measuring region. A sample capacity of the flow channel at the sampling region is larger than a sample capacity of the flow channel at the concentrating region and the measuring region. The first cover plate is disposed on the first surface and at least covers the flow channel. The first cover plate has a gas outlet. The gas outlet is near an end of the flow channel opposite to the sample inlet. The electrode assembly includes a first electrode pair, a second electrode pair, and a third electrode pair. The first electrode pair is located at a boundary between the sampling region and the concentrating region. The second electrode pair is located at a boundary between the concentrating region and the measuring region. The third electrode pair is located in the measuring region. The reactive unit is disposed on the third electrode pair and located in the flow channel.

According to an embodiment of the invention, the glucose measuring device further includes a processing unit. The processing unit is electrically connected to the electrode assembly.

According to an embodiment of the invention, the processing unit is disposed on the substrate, for example.

According to an embodiment of the invention, the glucose measuring device further includes a power supply unit. The power supply unit is electrically connected to the processing unit.

According to an embodiment of the invention, the glucose measuring device further includes a heating unit electrically connected to the power supply unit. The heating unit is disposed on the second surface and corresponds to the concentrating region.

According to an embodiment of the invention, the glucose measuring device further includes a second cover plate. The second cover plate is disposed on the second surface and at least covers the heating unit.

According to an embodiment of the invention, the power supply unit is disposed on the second cover plate and located between the second cover plate and the substrate, for example.

According to an embodiment of the invention, the glucose measuring device further includes a heating unit electrically connected to the power supply unit. The heating unit is disposed in the flow channel.

According to an embodiment of the invention, the reactive unit includes a conductive medium and an active substance capable of reacting with saliva.

According to an embodiment of the invention, the flow channel is located in the substrate, for example.

According to an embodiment of the invention, the flow channel is defined by a film layer disposed on the first surface, for example.

According to an embodiment of the invention, the glucose measuring device further includes a plurality of separators. The separators are disposed on sidewalls of the flow channel.

The invention provides a glucose measuring apparatus including a glucose measuring device and a detecting device. The glucose measuring device includes a substrate, a cover plate, an electrode assembly, and a reactive unit. The substrate has a first surface and a second surface opposite to each other, and a flow channel located at the first surface. The flow channel includes a sampling region having a sample inlet, a concentrating region, and a measuring region. The concentrating region is located between the sampling region and the measuring region. A sample capacity of the flow channel at the sampling region is larger than a sample capacity of the flow channel at the concentrating region and the measuring region. The cover plate is disposed on the first surface and at least covers the flow channel. The cover plate has a gas outlet. The gas outlet is near an end of the flow channel opposite to the sample inlet. The electrode assembly includes a first electrode pair, a second electrode pair, and a third electrode pair. The first electrode pair is located at a boundary between the sampling region and the concentrating region. The second electrode pair is located at a boundary between the concentrating region and the measuring region. The third electrode pair is located in the measuring region. The reactive unit is disposed on the third electrode pair and located in the flow channel. The detecting device is electrically connected to the glucose measuring device.

According to an embodiment of the invention, the detecting device is electrically connected to the electrode assembly of the glucose measuring device.

According to an embodiment of the invention, the detecting device includes a processing unit, a power supply unit, a heating unit, and a slot. The processing unit is electrically connected to the glucose measuring device. The power supply unit is electrically connected to the processing unit and the glucose measuring device. The heating unit is disposed at a position corresponding to the concentrating region of the glucose measuring device. The slot is electrically connected to the glucose measuring device.

According to an embodiment of the invention, the detecting device includes a gas outlet flue. The gas outlet flue is disposed on the gas outlet of the glucose measuring device. The gas outlet flue extends from the gas outlet in a direction away from the cover plate.

Based on the above, the glucose measuring device of the invention is used to measure the glucose concentration in the saliva of the subject, and thus it does not cause trauma to the subject and has higher accuracy. The measured value is comparable to the value of the glucose concentration measured in the blood.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is an explosion diagram illustrating a glucose measuring device according to an embodiment of the invention.

FIG. 2 is a schematic top view of a substrate in FIG. 1.

FIG. 3 is a schematic top view illustrating a substrate according to another embodiment of the invention.

FIG. 4A to FIG. 4D are schematic operation diagrams illustrating a glucose measuring device according to an embodiment of the invention.

FIG. 5 is a comparison result of cyclic voltammetry signals from the blood, the saliva stock solution, and the saliva concentrated by 10%, 30%, 50%, 70%, and 90% using the invention for the same subject.

FIG. 6 is a result of a linear regression analysis of the saliva and the blood respectively collected from a plurality of subjects and using the glucose measuring device of the invention and the commercially available blood glucose meter.

FIG. 7 is an explosion diagram of a glucose measuring apparatus having the glucose measuring device of the invention.

FIG. 8 is a schematic cross-sectional view of a gas outlet flue in the glucose measuring apparatus of the embodiment of the invention.

### DESCRIPTION OF THE EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

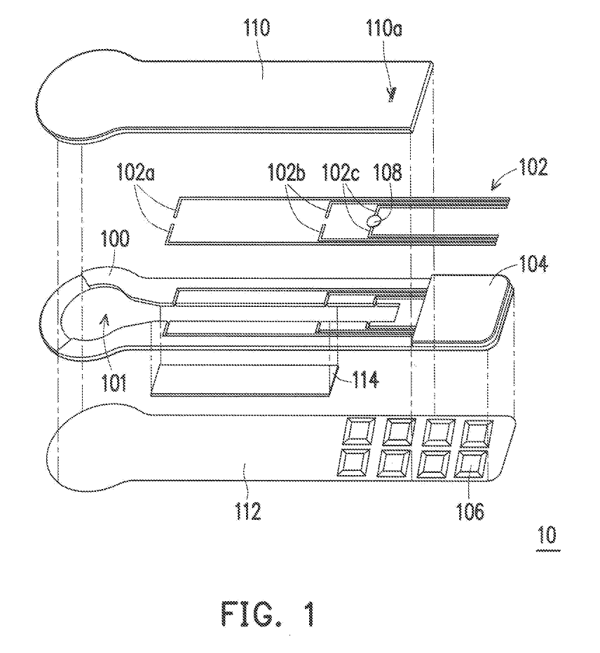

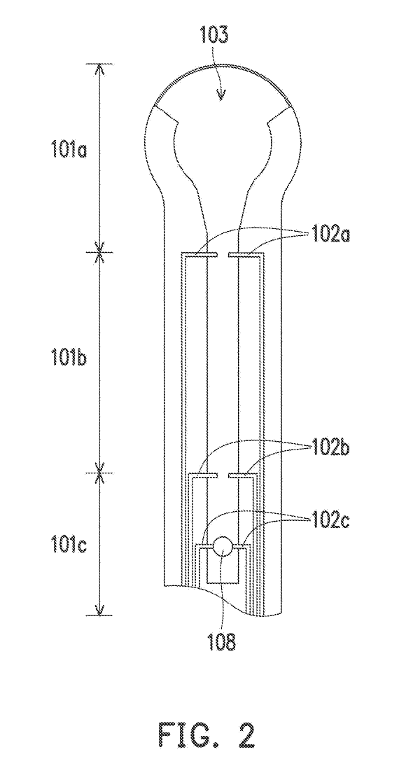

FIG. 1 is an explosion diagram illustrating a glucose measuring device according to an embodiment of the invention. FIG. 2 is a schematic top view of a substrate in FIG. 1. Referring to FIG. 1 and FIG. 2 at the same time, a glucose measuring device 10 includes a substrate 100, an electrode assembly 102, a processing unit 104, a power supply unit 106, a reactive unit 108, and cover plates 110 and 112. The cover plates 110 and 112 are respectively disposed at an upper side and a lower side of the substrate 100 and used to protect the substrate 100 and elements disposed on the substrate 100. Each component is further illustrated below.

A material of the substrate 100 is an electric insulating material, such as glass fiber, novolac resins, polycarbonate, acrylonitrile-butadiene-styrene (ABS) resins, melamine, glass, or ceramics. An upper surface of the substrate 100 has a flow channel 101. In the embodiment, the flow channel 101 can be formed in the substrate 100 directly in an injection molding process or an extrusion molding process for forming a main body of the substrate 100, or the flow channel 101 can be formed in the substrate 100 by performing a laser engraving process after forming the substrate 100. In other embodiments, the flow channel 101 can also be defined by a patterned film layer fon led on the main body of the substrate 100 after forming the main body of the substrate 100. That is, the flow channel 101 is located on a surface of the substrate 100. The aforementioned patterned film layer is a polypropylene (PP) adhesive tape, a polyvinyl chloride (PVC) adhesive tape, or a polyethylene terephthalate (PET) adhesive tape that the pattern of the flow channel 101 has been cut out, for example, or a heat drying type insulating paint or a UV curing type insulating paint formed by a printing method, for example.

The flow channel 101 includes a sampling region 101a, a concentrating region 101b, and a measuring region 101c. The concentrating region 101b is located between the sampling region 101a and the measuring region 101c. The sampling region 101a has a sample inlet 103 located at an edge of the substrate 100. The sample to be tested (saliva in the embodiment) may enter the flow channel 101 via the sample inlet 103. The sampling region 101a is used to accommodate a large number of the sample entering the flow channel 101 via the sample inlet 103. Also, to allow the sample to enter the concentrating region 101b and the measuring region 101c by capillary action, a sample capacity of the sampling region 101a is larger than a total sample capacity of the concentrating region 101b and the measuring region 101c. According to a direction of travel of the sample in the flow channel 101, the concentrating region 101b is located downstream of the sampling region 101a. In the concentrating region 101b, the sample can be concentrated to have a higher concentration. The measuring region 101c is located downstream of the concentrating region 101b. The measuring region 101c is used to measure the required sample parameters.

The electrode assembly 102 is disposed on the substrate 100. The state of the sample flowing through the flow channel 101 can be determined by an electrical signal difference provided by the electrode assembly 102 located at different regions of the flow channel 101. A material of the electrode assembly 102 may be any conductive material, such as a conductive paste. The conductive paste may be a palladium paste, a platinum paste, a gold paste, a titanium paste, a carbon paste, a silver paste, a copper paste, a mixed paste of gold and silver, a mixed paste of carbon and silver, or any combination of the above. Alternatively, the electrode assembly 102 may be composed of a conductive carbon powder layer or a metal layer. Alternatively, the electrode assembly 102 may be composed of a conductive paste and a conductive carbon powder layer located thereon, wherein an impedance of the conductive carbon powder layer is far more than that of the conductive paste.

Specifically, the electrode assembly 102 includes a first electrode pair 102a, a second electrode pair 102b, and a third electrode pair 102c. The first electrode pair 102a is located at a boundary between the sampling region 101a and the concentrating region 101b, which is used to determine whether sampling the sample is finished. Thus, the first electrode pair 102a can also be called as sampling electrodes. The second electrode pair 102b is located at a boundary between the concentrating region 101b and the measuring region 101c, which is used to determine whether the sample starts to be concentrated and determine whether the concentration has been finished. Thus, the second electrode pair 102b can also be called as concentrating electrodes. The third electrode pair 102c is located in the measuring region 101c, which is used to measure specific parameters in the concentrated sample. Thus, the third electrode pair 102c can also be called as measuring electrodes. However, the invention does not limit the use of each of the electrode pairs. In another embodiment, the first electrode pair 102a and the second electrode pair 102b may also have the function of parameter measurement.

The processing unit 104 is electrically connected to the electrode assembly 102, so as to analyze the parameters or the state of the sample through an electrical signal provided by the electrode assembly 102. Furthermore, the processing unit 104 is electrically connected to the first electrode pair 102a, so as to determine whether sampling the sample is finished through the electrical signal (e.g., impedance change, capacitive reactance change, or resistance change) provided by the first electrode pair 102a. The processing unit 104 is electrically connected to the second electrode pair 102b, so as to determine whether the concentration starts and deteiniine whether the concentration has been finished through the electrical signal (e.g., impedance change, capacitive reactance change, or resistance change) provided by the second electrode pair 102b. The processing unit 104 is electrically connected to the third electrode pair 102c, so as to measure the specific parameters in the concentrated sample through the electrical signal (e.g., a number of electrons) provided by the third electrode pair 102c. The processing unit 104 may be any processing unit having the aforementioned functions, and is not limited in the invention. Additionally, in the embodiment, the processing unit 104 is disposed on the substrate 100 and located at an end opposite to the sampling region 101a. In other embodiments, the processing unit 104 may also be disposed at any suitable position on the substrate 100, or the processing unit 104 may not be disposed on the substrate 100.

The power supply unit 106 is electrically connected to the processing unit 104, so as to provide the electrical power required for the processing unit 104 and the electrode assembly 102. The power supply unit 106 may be disposed at any suitable position in the glucose measuring device 10, and is not limited in the invention. In the invention, the type, the foilii, and the number of the power supply unit 106 are not limited in any way, as long as it can provide enough power to make the glucose measuring device 10 work. The power supply unit 106 is a printed battery, for example, and preferably is a printed micro-zinc battery.

The reactive unit 108 is disposed on the third electrode pair 102c and located in the flow channel 101, so as to contact and react with the sample flowing into the measuring region 101c. Specifically, the reactive unit 108 includes a conductive medium and an active substance capable of electrochemically reacting with the sample. In a condition that the sample is saliva, the aforementioned active substance, which may be an immobilized or non-immobilized enzyme (e.g., glucose oxidase or glucose dehydeogenase) may electrochemically react with the saliva. The conductive medium is used to receive electrons generated after the reaction of the active substance and the sample, and conduct the electrons to the processing unit 104 via the third electrode pair 102c, so as to measure the specific parameters in the concentrated sample. In the condition that the sample is the saliva, the aforementioned specific parameter is glucose concentration, for example. The conductive medium is red prussiate, thionine, phenazine methosulfate, potassium ferrocynaide, or methyl viologen, for example. Additionally, the reactive unit 108 may further include other additives, such as a buffer solution or a protective agent (e.g., protein, dextrin, dextran, or amino acid).

The cover plate 110 is disposed at an upper side of the substrate 100 and used to cover the flow channel 101. As shown in FIG. 1, the cover plate 110 covers the sampling region 101a, the concentrating region 101b, and the measuring region 101c of the flow channel 101, but the sample inlet 103 is not closed, such that the sample can enter the flow channel 101 via the sample inlet 103. Additionally, the cover plate 110 has a gas outlet 110a. The gas outlet 110a is located near an end of the flow channel 101 opposite to the sample inlet 103. The gas outlet 110a is used to exhaust the gas in the flow channel 101, so as to enhance the capillary action of the sample after entering the flow channel 101. The shape of the gas outlet 110a is not limited in the invention. For example, the gas outlet 110a may be circular, oval, rectangular, or rhombic. In an embodiment, a surface of the cover plate 110 near the flow channel 101 may have a hydrophilic coating (not shown) thereon to further reduce flow resistance of the sample in the flow channel 101 and enhance the capillary action in the flow channel 101, such that the sample can be quickly and effectively introduced into the flow channel 101.

The cover plate 112 is disposed at a lower side of the substrate 100. In the embodiment, the power supply unit 106 is disposed on the cover plate 112 and located between the cover plate 112 and the substrate 100. Therefore, the cover plate 112 can protect the power supply unit 106 from damage.

Additionally, in the embodiment, the glucose measuring device 10 may further optionally include a heating unit 114. The heating unit 114 is disposed at the lower side of the substrate 100 and corresponds to the concentrating region 101b, and is electrically connected to the power supply unit 106. The heating unit 114 may also be covered by the cover plate 110 without damage. The heating unit 114 is used to heat the sample flowing through the concentrating region 101b, such that the water in the sample is evaporated to achieve the purpose of concentrating the sample. The heating unit 114 is an electrically heated wire, a graphite sheet, or a heat conductive silicone sheet, for example. In another embodiment, the heating unit 114 may also be directly disposed in the flow channel 101. At this time, the heating unit 114 is a heating wire disposed on inner walls of the flow channel 101, for example. In other embodiments, the glucose measuring device 10 may not be provided with the heating unit 114, and the purpose of concentrating the sample is achieved by that the water of the sample is naturally evaporated to the air in the natural environment.

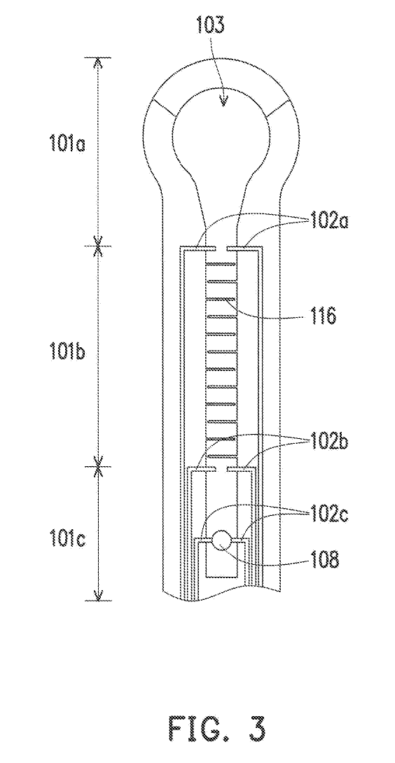

To increase flow distance and flow time of the sample in the flow channel 101 to increase the heating time of the sample, a separator 116 may be disposed on the sidewalls of the flow channel 101 as shown in FIG. 3. The separator 116 may be formed integrally with the flow channel 101, or additionally disposed on the sidewalls of the flow channel 101. In a condition that the heating unit 114 is directly disposed in the flow channel 101, the heating unit 114 may be disposed along the the separator 116 and inner walls of the flow channel 101.

Additionally, the cover plate 112 may be omitted depending on whether the heating unit 114 is used and the position thereof, and the position of the power supply unit 106.

It should be mentioned that the glucose measuring device 10 may also include other additional components depending on the actual needs. For example, the glucose measuring device 10 may include a display unit used to display measurement results and prompt the subjects. The position of the display unit is not limited in the invention. For example, the display unit may be disposed above the cover plate 110 or/and the processing unit 104, or may be disposed below the cover plate 112. The display unit may be a bi-stable display. Additionally, the glucose measuring device 10 may also include a prompt unit used to info ni the subjects that the sampling is completed, the test is finished, or other states. The aforementioned additional components can be disposed at suitable positions depending on the actual needs, and are not limited in the invention.

The operation of the glucose measuring device of the invention will be described below with reference to the glucose measuring device 10 as an example.

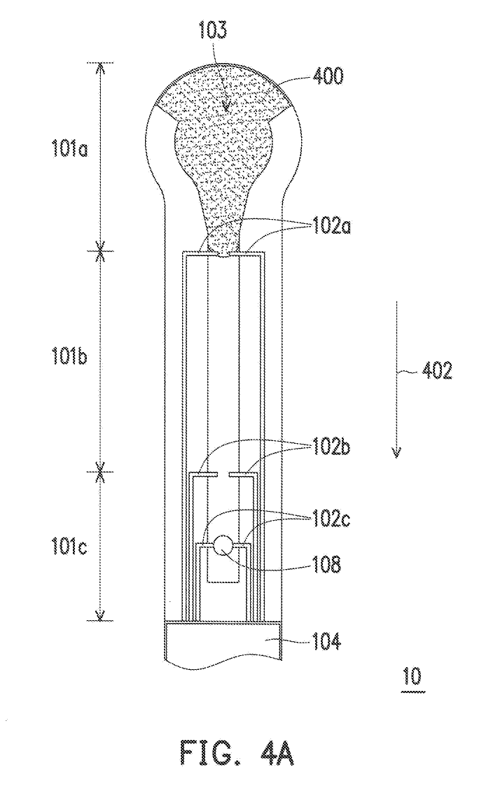

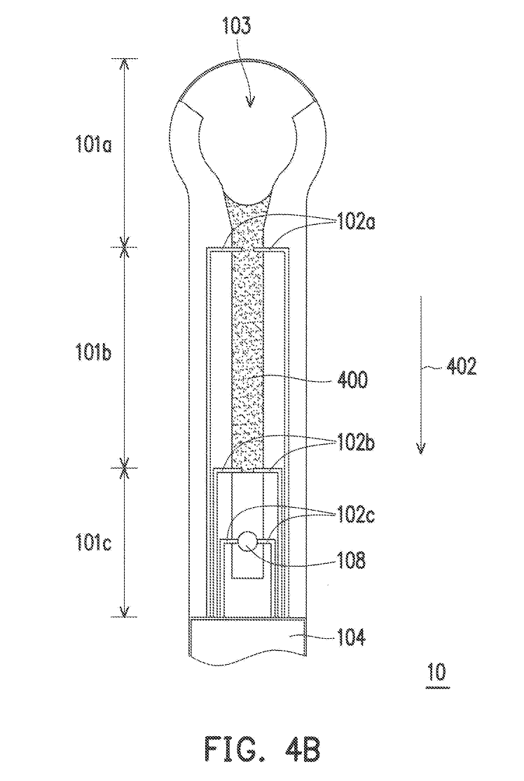

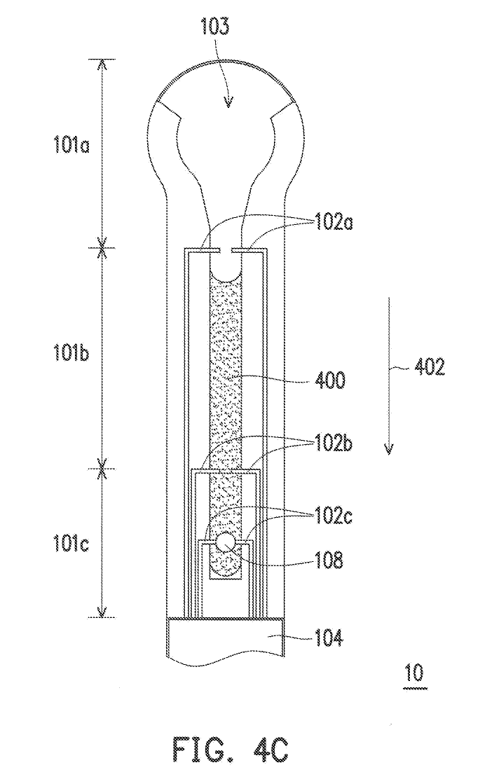

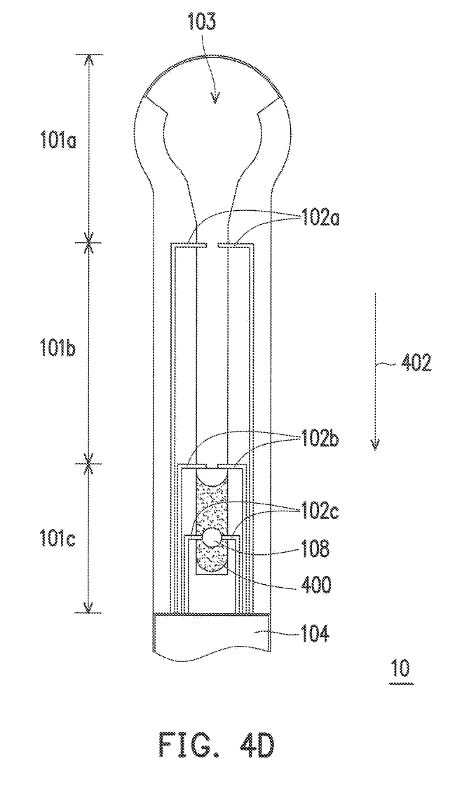

FIG. 4A to FIG. 4D are schematic operation diagrams illustrating a glucose measuring device according to an embodiment of the invention. In FIG. 4A to FIG. 4D, for clarity, parts of components are omitted, and it is described by the substrate in the schematic top view.

First, referring to FIG. 4A, the subject puts the glucose measuring device 10 into the mouth, such that saliva 400 enters the flow channel 101 from the sample inlet 103. At this time, the sampling region 101a is filled with the saliva 400 due to the capillary action, and the saliva 400 flows along a direction of an arrow 402. When the saliva 400 flows through the first electrode pair 102a electrically connected to the power supply unit 106, the processing unit 104 can determine that the saliva 400 has entered the concentrating region 101b by the electrical signal difference generated from the impedance change, capacitive reactance change, or resistance change caused by the saliva 400. Additionally, since the sample capacity of the sampling region 101a is larger than the total sample capacity of the concentrating region 101b and the measuring region 101c, the processing unit 104 may also determine that the sampling is enough to prompt the subject to stop sampling. In a condition that the glucose measuring device 10 includes the display unit, the display unit can be used to infoini the subject to stop sampling. In a condition that the glucose measuring device 10 includes the prompt unit, the prompt unit can be used to inform the subject to stop sampling by sending out a voice prompt or light prompt.

Then, referring to FIG. 4B, the saliva 400 continues to flow along the direction of the arrow 402 by the capillary action. When the saliva 400 flows to the second electrode pair 102b electrically connected to the power supply unit 106, the processing unit 104 can determine that the concentrating region 101b has been filled with the saliva 400 by the electrical signal difference generated from the impedance change, capacitive reactance change, or resistance change caused by the saliva 400. In the embodiment, when the processing unit 104 determines that the concentrating region 101b has been filled with the saliva 400, the processing unit 104 activates the heating unit 114 at the same time, so as to provide the saliva 400 in the concentrating region 101b with thermal energy to evaporate water in the saliva 400. Thereby, a volume of the saliva 400 is changed to achieve the purpose of concentrating. The water vapor generated by evaporation of water can be exhausted via the gas outlet 110a. The heating temperature and the heating time are not limited in the invention, as long as the heating unit 114 can provide enough thermal energy to change the volume of the saliva 400. In an embodiment, the heating temperature is between 20° C. and 50° C., for example. The volume of the saliva 400 after concentrating is between 20% and 90% of an original volume, for example.

Then, referring to FIG. 4C, the saliva 400 continues to flow along the direction of the arrow 402 by the capillary action, so as to fill in the measuring region 101c. Although the volume of the saliva 400 in the concentrating region 101b is changed, the capillary action in the flow channel 101 is still continued. Thus, the concentrated saliva 400 still continues to flow along the direction of the arrow 402 until the volume of the saliva 400 is less than the volume of the measuring region 101c.

Thereafter, referring to FIG. 4D, when the volume of the saliva 400 is less than the volume of the measuring region 101c, the second electrode pair 102b is changed from the state of being in contact with the saliva 400 to the state of not being in contact with the saliva 400 to cause the impedance change, capacitive reactance change, or resistance change. Thus, the electrical signal difference is generated again. At this time, the processing unit 104 can determine that the concentration has been finished through the electrical signal difference and the glucose concentration is measure using the third electrode pair 102c. The reactive unit 108 on the third electrode pair 102c is in contact with and react with the saliva 400. The electrons generated after the reaction are conducted to the processing unit 104 via the third electrode pair 102c, such that the glucose concentration in the concentrated saliva 400 is measured. At this time, since the saliva 400 has been concentrated, the glucose concentration in the saliva 400 is increased. Thereby, the measurement signals are increased. Therefore, the accuracy of the measured values can be comparable to the value of the glucose concentration measured in the blood. Additionally, the measurement of the glucose concentration in the body of the subject in the aforementioned manner does not cause trauma to the subject. That is, the glucose measuring device 10 of the invention is a noninvasive glucose measuring device.

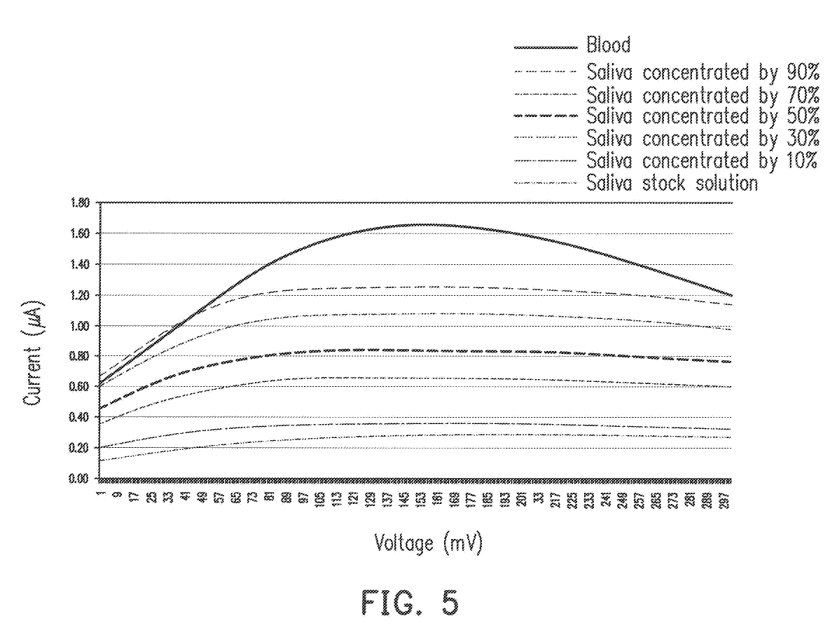

FIG. 5 is a comparison result of cyclic voltammetry signals from the blood, the saliva stock solution, and the saliva concentrated by 10%, 30%, 50%, 70%, and 90% using the invention for the same subject. Those skilled in the art have known that the cyclic voltammetry detection is to perform potential scanning on the sample. The potential scanning can be used for the sample redox signal analysis. As shown in FIG. 5, a peak value signal measured from the saliva stock solution is about 0.23 μA. A peak value signal measured from the saliva concentrated by 10% is about 0.32 μA. A peak value signal measured from the saliva concentrated by 30% is about 0.65 μA. A peak value signal measured from the saliva concentrated by 50% is about 0.82 μA. A peak value signal measured from the saliva concentrated by 70% is about 1.14 μA. A peak value signal measured from the saliva concentrated by 90% is about 1.22 μA. A peak value signal measured from the blood is about 1.63 μA. It is clear from FIG. 5 that the measurement signals of the concentrated saliva are significantly increased, and the linear response thereof is close to the measurement result of the blood.

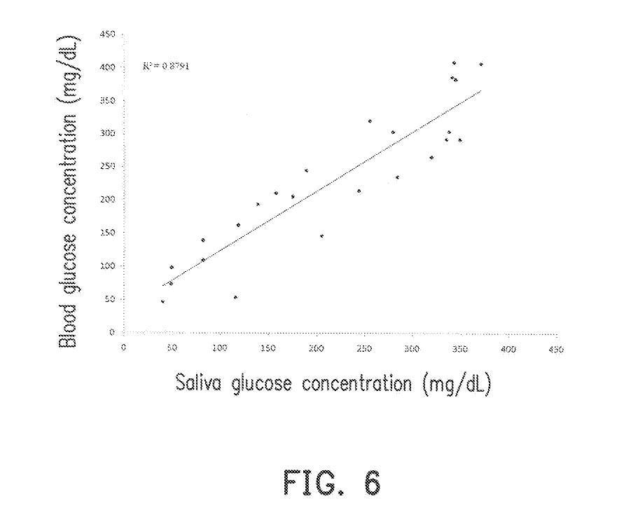

FIG. 6 is a result of a linear regression analysis of the blood and the saliva respectively collecting from a plurality of subjects and using the glucose measuring device of the invention and the commercially available blood glucose meter. Those skilled in the art have known that the linear regression analysis is to analyze the correlation of the measurement results on two systems (the glucose measuring device of the invention and the commercially available blood glucose meter), wherein the closer the analyzing data R² is to 1, the closer the measurement results of the two systems are. In the experiment, the eight blood glucose concentration ranges of 50 mg/dL to 99 mg/dL, 100 mg/dL to 149 mg/dL, 150 mg/dL to 199 mg/dL, 200 mg/dL to 249 mg/dL, 250 mg/dL to 299 mg/dL, 300 mg/dL to 349 mg/dL, 350 mg/dL to 399 mg/dL, and 400 mg/dL to 449 mg/dL are respectively collected. The saliva and the blood of three subjects in each of the concentration range are collected (total 24 test samples), and then the concentrations thereof are respectively detected using the glucose measuring device of the invention and the commercially available blood glucose meter. As shown in FIG. 6, the measurement correlation R² using the glucose measuring device of the invention and the commercially available blood glucose meter is 0.8387, and each data is not significantly dispersed. Thus, it is confirmed that the glucose measuring device of the invention has the accuracy meeting the needs.

It should be mentioned that, when measuring the glucose concentration in the blood, the measurement results usually have about 20% of error due to hemotocrit (HCT). However, since the saliva does not have the aforementioned interference factor (hemotocrit), the accuracy of the measured values from the concentrated saliva is comparable to the accuracy of the value of the glucose concentration measured in the blood even though the peak value signal measured from the concentrated saliva is lower than the peak value signal measured from the blood in FIG. 5.

It should be mentioned that a height of the sample inlet may be higher than a height of the gas outlet to ensure the flow of the saliva and increase the exclusion of the water vapor. That is, the sample inlet and the gas outlet are at a non-horizontal angle. The aforementioned non-horizontal angle may be between 5 degrees and 90 degrees, and preferably between 20 degrees and 50 degrees. The height of the sample inlet being higher than the height of the gas outlet may be made by forming the glucose measuring device of the invention to a non-horizontal structure, or when using the glucose measuring device of the invention, it is used in an inclined angle.

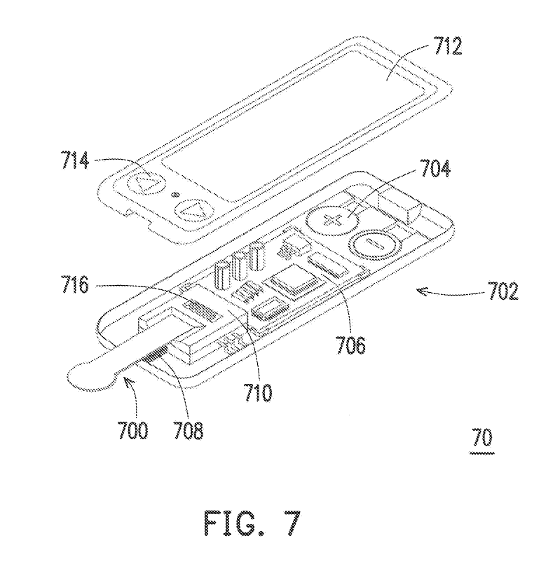

FIG. 7 is an explosion diagram of a glucose measuring apparatus having the glucose measuring device of the invention. Refening to FIG. 7, a glucose measuring apparatus 70 includes a glucose measuring device 700 (without the processing unit 104, the power supply unit 106, and the heating unit 114 in FIG. 1) similar to the glucose measuring device 10 and a detecting device 702. In the embodiment, since the glucose measuring device 700 does not include the processing unit 104 in FIG. 1, the exposed electrode assembly 102 can be used as a connector electrically connected to an outer device, and the detecting device 702 is electrically connected to the glucose measuring device 700 via the connector.

The detecting device 702 includes a power supply unit 704, a processing unit 706, a heating unit 708, and a slot 710. The slot 710 is used to be electrically connected to the glucose measuring device 700, such that the detecting device 702 can provide the power to and detect the electrical signal from the glucose measuring device 700 via the slot 710. The heating unit 708 is disposed at the position corresponding to the concentrating region 101b of the glucose measuring device 700 to heat the sample flowing through the concentrating region 101b, so as to achieve the purpose of concentrating the sample. The processing unit 706 analyzes the parameters or the state of the sample through the received electrical signal. The power supply unit 704 provides the processing unit 706 and the glucose measuring device 700 with the required power. The positions of the power supply unit 704, the processing unit 706, the heating unit 708, and the slot 710 are not particularly limited in the invention, and can be adjusted depending on the actual needs.

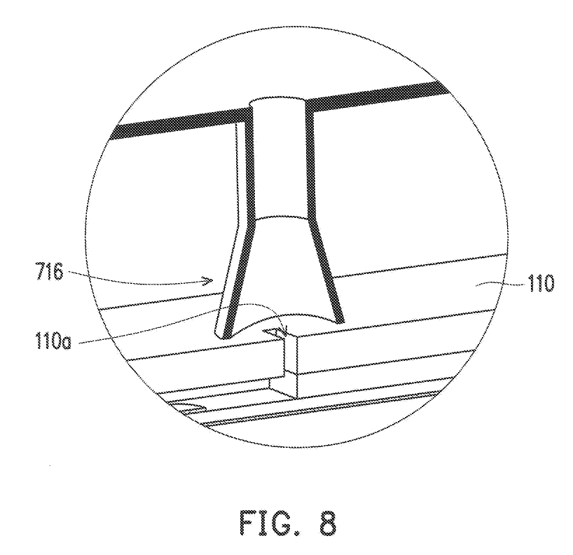

In an embodiment, the detecting device 702 includes a gas outlet flue 716. As shown in FIG. 8, the gas outlet flue 716 is located on the gas outlet 110a of the glucose measuring device 700, and the gas outlet flue 716 extends from the gas outlet 110a in a direction away from the cover plate 110. When the water vapor generated by heating the concentrated sample is exhausted from the gas outlet 110a of the glucose measuring device 700, the water vapor is exhausted out of the detecting device 702 along the gas outlet flue 716 to prevent the detecting device 702 from being damaged due to moisture. Additionally, to avoid the water vapor attaching on inner tube walls of the gas outlet flue 716 in the process of exhausting and then refluxing into the detecting device 702, the inner tube walls of the gas outlet flue 716 of the embodiment may have a hydrophobic effect. For example, the tube walls of the gas outlet flue 716 may be made by the material with the hydrophobic effect, or a hydrophobic layer may be disposed on the inner tube walls of the gas outlet flue 716. The structure of the gas outlet flue 716 is not limited in the embodiment, as long as it has an effect of guiding the water vapor to be exhausted. In the embodiment, a diameter of the water vapor inlet (near the opening of the gas outlet 110a) of the gas outlet flue 716 is larger than a diameter of the outlet (i.e., the opening far away from the gas outlet 110a), and the appearance of the gas outlet flue 716 is conical. However, the invention is not limited thereto. In other embodiments, the gas outlet flue 716 may have other appearances and structures depending on the actual needs.

Additionally, the glucose measuring apparatus 70 may also include other devices depending on the actual needs, such as a display unit 712 used to display an image, measurement results, steps, and other parameter values, and an operating unit 714 used to provide the users to perform interface switching and operation setting. However, the invention is not limited thereto. Additionally, the glucose measuring apparatus 70 may also be provided with a password card (not shown), which includes one or more sets of parameter values to correct various parameters (e.g., magnifying power, slope, intercept, temperature/humidity compensation coefficient, or test piece valid date) of the glucose measuring apparatus 70.

Additionally, in other embodiments, the glucose measuring device in the glucose measuring apparatus may include at least one of the processing unit, the power supply unit, and the heating unit depending on the actual needs. At this time, the detecting device in the glucose measuring apparatus does not have the aforementioned elements.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A glucose measuring device, suitable for measuring glucose in saliva, comprising:
a substrate, having a first surface and a second surface opposite to each other, and a flow channel located at the first surface, the flow channel comprising a sampling region having a sample inlet, a concentrating region, and a measuring region, wherein the concentrating region is located between the sampling region and the measuring region, and a sample capacity of the flow channel at the sampling region is larger than a sample capacity of the flow channel at the concentrating region and the measuring region;
a first cover plate, disposed on the first surface and at least covering the flow channel, the first cover plate having a gas outlet, and the gas outlet being near an end of the flow channel opposite to the sample inlet;
an electrode assembly, comprising a first electrode pair, a second electrode pair, and a third electrode pair, the first electrode pair being located at a boundary between the sampling region and the concentrating region, the second electrode pair being located at a boundary between the concentrating region and the measuring region, and the third electrode pair being located in the measuring region; and
a reactive unit, disposed on the third electrode pair and located in the flow channel.

2. The glucose measuring device according to claim 1, further comprising a processing unit, the processing unit being electrically connected to the electrode assembly.

3. The glucose measuring device according to claim 2, wherein the processing unit is disposed on the substrate.

4. The glucose measuring device according to claim 2, further comprising a power supply unit, the power supply unit being electrically connected to the processing unit.

5. The glucose measuring device according to claim 4, further comprising a heating unit electrically connected to the power supply unit, the heating unit being disposed on the second surface and corresponding to the concentrating region.

6. The glucose measuring device according to claim 5, further comprising a second cover plate, the second cover plate being disposed on the second surface and at least covering the heating unit.

7. The glucose measuring device according to claim 6, wherein the power supply unit is disposed on the second cover plate and located between the second cover plate and the substrate.

8. The glucose measuring device according to claim 4, further comprising a heating unit electrically connected to the power supply unit, the heating unit being disposed in the flow channel.

9. The glucose measuring device according to claim 1, wherein the reactive unit comprises a conductive medium and an active substance capable of reacting with the saliva.

10. The glucose measuring device according to claim 1, wherein the flow channel is located in the substrate.

11. The glucose measuring device according to claim 1, wherein the flow channel is defined by a film layer disposed on the first surface.

12. The glucose measuring device according to claim 1, further comprising a plurality of separators, the separators being disposed on sidewalls of the flow channel.

13. A glucose measuring apparatus, suitable for measuring glucose in saliva, comprising:
a glucose measuring device, comprising:
a substrate, having a first surface and a second surface opposite to each other, and a flow channel located at the first surface, the flow channel comprising a sampling region having a sample inlet, a concentrating region, and a measuring region, wherein the concentrating region is located between the sampling region and the measuring region, and a sample capacity of the flow channel at the sampling region is larger than a sample capacity of the flow channel at the concentrating region and the measuring region;
a cover plate, disposed on the first surface and at least covering the flow channel, the cover plate having a gas outlet, the gas outlet being near an end of the flow channel opposite to the sample inlet;
an electrode assembly, comprising a first electrode pair, a second electrode pair, and a third electrode pair, the first electrode pair being located at a boundary between the sampling region and the concentrating region, the second electrode pair being located at a boundary between the concentrating region and the measuring region, and the third electrode pair being located in the measuring region; and
a reactive unit, disposed on the third electrode pair and located in the flow channel; and
a detecting device, electrically connected to the glucose measuring device.

14. The glucose measuring apparatus according to claim 13, wherein the detecting device is electrically connected to the electrode assembly of the glucose measuring device.

15. The glucose measuring apparatus according to claim 13, wherein the detecting device comprises:
a processing unit, electrically connected to the glucose measuring device;
a power supply unit, electrically connected to the processing unit and the glucose measuring device;
a heating unit, disposed at a position corresponding to the concentrating region of the glucose measuring device; and
a slot, electrically connected to the glucose measuring device.

16. The glucose measuring apparatus according to claim 13, wherein the detecting device comprises a gas outlet flue, the gas outlet flue is disposed on the gas outlet of the glucose measuring device, and the gas outlet flue extends from the gas outlet in a direction away from the cover plate.
